(19) United States
(12) Patent Application Publication
(10) Pub. No.: US 2006/0106268 A1
(43) Pub. Date: May 18, 2006

(54) High activity and high stability iron oxide based dehydrogenation catalyst having a low concentration of titanium and the manufacture and use thereof

(75) Inventor: Ruth Mary Kowaleski, Cypress, TX (US)

Correspondence Address: Shell Oil Company, 910 Louisiana, Houston, TX, 77002, US

(73) Assignee: Shell Oil Company

(21) Appl. No.: 11/280,578

(22) Filed: Nov. 16, 2005

Related U.S. Application Data
(60) Provisional application No. 60/628,996, filed on Nov. 18, 2004.

(51) Int. Cl. C07C 2/64; C01G 49/02

(52) U.S. Cl. 585/444; 423/632; 502/316; 502/338

(57) ABSTRACT

A high activity and high stability dehydrogenation catalyst, its manufacture and its use. The catalyst comprises an iron ox:de component having a low titanium content, wherein the iron oxide component is made by the heat-treating of a yellow iron oxide precipitate made by precipitating from a solution of an iron salt the yellow iron oxide precipitate and, optionally, an additional dehydrogenation catalytic component, wherein the iron oxide based dehydrogenation catalyst composition has a low concentration of titanium. The low titanium containing iron oxide based dehydrogenation catalyst is used in the dehydrogenation of dehydrogenatable hydrocarbons.

This application claims the benefit of U.S. Provisional Application No. 60/628,996 filed Nov. 18, 2004, the entire disclosure of which is hereby incorporated by reference.

The invention relates to an iron oxide based dehydrogenation catalyst having a low concentration of titanium and the manufacture and use thereof.

In the field of catalytic dehydrogenation of alkylaromatic hydrocarbons to alkenylaromatic hydrocarbons there are ongoing efforts to develop improved catalysts that have properties of high activity and selectivity while exhibiting high stability when in use. The stability of a catalyst refers to its rate of catalytic deactivation or decline when in use. The rate of deactivation of a catalyst impacts its useful life, and, generally, it is preferable for a catalyst to be highly stable so as to increase its useful life and to improve product yields at lower severity process conditions.

Iron oxide based catalysts have commonly been used in the dehydrogenation of alkylaromatic compounds, such as ethylbenzene, to yield corresponding alkenylaromatic compounds, such as styrene. These iron oxide based catalysts may be formulated using a variety of sources and forms of iron oxide including, for example, yellow iron oxide (goethite, FeOOH), black iron oxide (magnetite, Fe₃O₄), and red iron oxide (hematite, Fe₂O₃), including synthetic hematite or regenerator iron oxide.

One type of iron oxide that is a possible candidate for use as a component of iron oxide based dehydrogenation catalysts may include those iron oxides produced by the spray roasting of an iron chloride solution. An example of a process for the production of such an iron oxide is disclosed in U.S. Pat. No. 5,911,967, which describes a process for the production of iron oxides by the spray roasting of an iron chloride containing solution within a reaction chamber wherein the solution is thermally decomposed to yield iron oxide.

Restructured iron oxide, when used in the preparation of dehydrogenation catalysts, has been found to provide for enhanced catalytic selectivity. U.S. Pat. No. 5,668,075 discloses a method of making restructured iron oxide that is a particularly desirable component for use in forming iron oxide based dehydrogenation catalysts. U.S. Pat. No. 5,668,075 discloses the preparation of restructured iron oxide by contacting or combining an effective amount of a restructuring agent with starting iron oxide material and then heating the resulting mixture until restructuring occurs. The resulting restructured iron oxide may suitably be used in the preparation of iron oxide based dehydrogenation catalysts.

U.S. Patent Publication US2003/0144566 discloses an iron oxide based dehydrogenation catalyst that is obtained by combining an iron oxide obtained by heat decomposition of an iron halide, such as that disclosed in U.S. Pat. No. 5,911,967, and yellow iron oxide. The combination or mixture may be calcined to yield a final catalyst composition. The patent publication also indicates that minor amounts of other types of iron oxides may be combined with the iron oxide obtained by heat decomposition of an iron halide. One such example is Penniman red iron oxide such as that made by the Penniman method taught by U.S. Pat. No. 1,368,748. Other examples include goethite, hematite and magnetite, as earlier mentioned above, and maghemite and lepidocricite.

As for the use of the so-called Penniman red iron oxide as a component of iron oxide based dehydrogenation catalysts, U.S. Pat. No. 5,689,023 discloses the preferred use of large particle iron oxide formulated from iron oxide derived from scrap iron via dehydration of yellow α-FeOOH intermediate, or, in other words, iron oxide derived from yellow iron oxide prepared by the Penniman method.

U.S. Pat. No. 5,190,906 teaches the addition of titanium oxide to an iron oxide based dehydrogenation catalyst for the purpose of enhancing the catalytic performance of the catalyst. The '906 patent asserts that the addition of from 0.005 to 0.95 wt. % (50 ppmw to 9500 ppmw) titanium oxide to the mixture of catalytic components of an iron oxide based dehydrogenation catalyst improves the performance of the catalyst. The '906 patent further indicates that the iron oxide used in forming its catalyst may be from a number of different sources including iron oxide produced by the precipitation method. When such iron oxide is used, the titanium oxide may be incorporated into the catalyst by the addition of water-soluble titanium salts into the aqueous iron salt solution used in the manufacture of iron oxide and iron oxide precursors.

As noted in the aforedescribed prior art, Penniman red iron oxide or precursors of red iron oxide produced by the Penniman precipitation method may be desirable sources of iron oxide for use in the formulation of iron oxide based dehydrogenation catalysts. There are continuing efforts to use such sources of iron oxide in the development of dehydrogenation catalyst compositions that have improved catalytic properties over the prior art catalysts.

It is, therefore, an object of this invention to provide an iron oxide based dehydrogenation catalyst having improved catalytic properties.

It is another object of the invention to provide a dehydrogenation catalyst that utilizes as a component thereof an iron oxide or iron oxide precursor derived by a precipitation method.

It is yet another object of the invention to provide a method of making an iron oxide based dehydrogenation catalyst that utilizes an iron oxide or iron oxide precursor made by a precipitation method.

Still another object of the invention is to provide an improved dehydrogenation process that utilizes an iron oxide based dehydrogenation catalyst prepared with an iron oxide or iron oxide precursor derived by a precipitation method.

Accordingly, one invention is an iron oxide based dehydrogenation catalyst composition that comprises an iron oxide component having a low titanium content, wherein the iron oxide component is made by heat-treating of a yellow iron oxide precipitate made by precipitating from a solution of an iron salt the yellow iron oxide precipitate, and wherein the iron oxide based dehydrogenation catalyst composition has a first concentration of titanium. The iron oxide based dehydrogenation catalyst composition may further include an additional dehydrogenation catalyst component.

Another invention includes a method of making an iron oxide based dehydrogenation catalyst having a first concentration of titanium. The method comprises forming a red iron oxide component, having a low titanium content, by heat-treating a yellow iron oxide precipitate made by the precipitation thereof from a solution of an iron salt, wherein the yellow iron oxide has a second concentration of titanium. The red iron oxide component is mixed with an additional dehydrogenation catalyst component and water to form a mixture from which a particle is formed, and heat-treating the particle to thereby provide the iron oxide based dehydrogenation catalyst.

Yet, another invention includes a dehydrogenation process, comprising: contacting under dehydrogenation conditions a dehydrogenatable hydrocarbon with an iron oxide based dehydrogenation catalyst composition and yielding a dehydrogenation product. The iron oxide based dehydrogenation catalyst composition comprises an iron oxide component having a low titanium content, wherein the iron oxide component is made by heat-treating of a yellow iron oxide precipitate made by precipitating from a solution of an iron salt the yellow iron oxide precipitate, wherein the iron oxide based dehydrogenation catalyst composition has a first concentration of titanium. The iron oxide based dehydrogenation catalyst may further include an additional dehydrogenation catalyst component.

In yet another invention, provided is a method of improving the operation of a dehydrogenation reactor system which comprises a dehydrogenation reactor containing a first volume of a dehydrogenation catalyst having an activity reducing concentration of titanium. This method includes removing from the dehydrogenation reactor the dehydrogenation catalyst and replacing therewith an iron oxide based dehydrogenation catalyst composition, which comprises an iron oxide component having a low titanium content and an additional dehydrogenation catalyst component, to thereby provide a second dehydrogenation reactor system. The iron oxide component is made by heat-treating a yellow iron oxide precipitate made by precipitating from a solution of an iron salt the yellow iron oxide precipitate. The second dehydrogenation reactor system may then be operated under dehydrogenation reaction conditions.

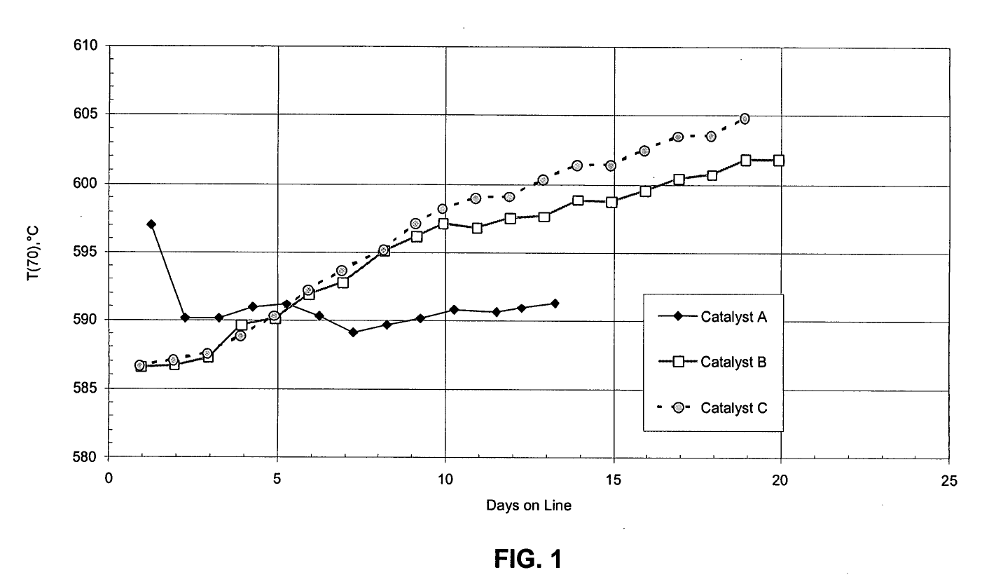

FIG. 1 presents plots of the dehydrogenation activity as a function of time for two dehydrogenation catalysts with one having a high titanium content and the other having a low titanium content.

It has been discovered that certain iron oxide based dehydrogenation catalyst formulations having low concentrations of titanium exhibit improved catalytic properties over certain other iron oxide based dehydrogenation catalyst formulations having higher concentrations of titanium. But, in the U.S. Pat. No. 5,190,906 mentioned above, it is taught that the addition of titanium dioxide to an iron oxide and potassium oxide catalyst system provides for an improvement in its catalytic properties such as activity, selectivity and stability. The '906 patent further teaches that when the source of the iron oxide component is from a precipitation process the titanium oxide may be added to the iron oxide before it is formed into catalyst particles by adding a water-soluble titanium salt to the aqueous iron salt solution from which iron oxide is obtained by precipitation. Contrary to these teachings, however, it has been found that the use as a component of an iron oxide based dehydrogenation catalyst of iron oxide having a significant titanium component and obtained by the aforementioned precipitation method provides a dehydrogenation catalyst having less desirable, rather than improved, catalytic properties.

The catalyst of the invention is an iron oxide based dehydrogenation catalyst that is useful in the dehydrogenation of dehydrogenatable hydrocarbons. The inventive catalyst exhibits improved stability over certain other iron oxide based dehydrogenation catalysts that contain higher concentrations of titanium as compared to the concentration of titanium of the inventive catalyst.

What is meant when referring herein to the stability of a catalyst is its rate of catalytic deactivation or decline when in use. Thus, the term stability refers to the rate at which a dehydrogenation catalyst deactivates expressed in terms of the ratio of the change in catalyst activity for a given time period of use of the catalyst at specific reaction conditions (Δ activity per Δ time). It is recognized that the rate at which a catalyst deactivates depends greatly upon the reaction conditions under which the catalyst is utilized, and, so, when comparing the stability of different catalysts, it is appropriate to compare their stability properties when used under the same or similar process conditions.

References herein to the activity of a catalyst are meant to refer to the temperature parameter associated with the particular catalyst. In the case of a styrene manufacturing catalyst, its temperature parameter is the temperature, in ° C., at which the styrene manufacturing catalyst provides under certain defined process conditions a specified conversion of an ethylbenzene feed. An illustrative example of activity is the temperature at which a conversion of 70 mole % of the ethylbenzene is achieved when contacted under certain specified reaction conditions. Such a temperature parameter may be represented by the symbol “T(70)”, which means that the given temperature provides for a conversion of 70 mole percent. The T(70) temperature value represents the activity of the related catalyst with the activity of a catalyst being inversely related to the temperature parameter where higher activities are represented by lower temperature parameters and lower activities are represented by higher temperature parameters.

As used herein, the term “conversion” means the fraction, in mole %, of a specified compound converted to another compound.

The term “selectivity” means herein the fraction, in mole %, of the converted compound that yields the desired compound.

One feature of the inventive iron oxide based dehydrogenation catalyst composition is that it is characterized as having a low titanium content. It is believed that the combination of the use as a catalyst component of a particular type of red iron oxide that is derived from a yellow iron oxide precipitate formed from a solution of an iron salt and the low titanium content of the yellow iron oxide precipitate gives the finished catalyst of the invention its improved stability properties.

The iron oxide component of the inventive catalyst can be derived by the heat treatment of a yellow iron oxide precipitate prepared by any suitable method known to those skilled in the art; provided, that, the yellow iron oxide precipitate that is heat treated to form the iron oxide component contains a sufficiently low titanium content. It is preferred for the yellow iron oxide to be made by any of the known Penniman or modified Penniman processes for the manufacture of yellow iron oxide. Such processes are described in such exemplary patents as U.S. Pat. No. 1,327,061; U.S. Pat. No. 1,368,748; U.S. Pat. No. 2,127,907; and U.S. Pat. No. 5,032,180, all of which are incorporated herein by reference. Thus, the yellow iron oxide precipitate that is heat treated to yield the iron oxide component of the inventive catalyst is, in general, prepared by a method, which includes introducing metallic iron into a solution of an iron salt, introducing into the solution an oxidizing agent such are oxygen, and yielding a yellow iron oxide precipitate. The metallic iron that is introduced or immersed into the solution of iron salt may be and often is scrap iron that is suspended within the solution. To further assist in the formation of the yellow iron oxide precipitate, a nucleus or seed particle, such as a colloidal hydrate of iron may be added to the solution.

One of the problems with the use of the yellow iron oxide prepared by the aforementioned precipitation, or Penniman, process as the precursor of the red iron oxide component of the finished iron oxide based dehydrogenation catalyst is that the raw materials used in its manufacture may contain impurities that negatively affect the performance of the finished iron oxide based dehydrogenation catalyst. In particular, the metallic iron used in the solution of iron salt of the Penniman process can contain such high levels of titanium that its use will result in providing a yellow iron oxide precipitate having an undesirably high titanium content. Also, other of the raw materials used in the Penniman process, such as the iron salts used to form the aqueous solution and the iron hydrates used as seeding material can have significant enough levels of titanium that the resulting precipitated yellow iron oxide will have an undesirably high level or concentration of titanium.

It is, therefore, a significant and important feature of the invention for the yellow iron oxide precipitate used as a precursor of the red iron oxide component of the inventive catalyst to have a low titanium content. In order to provide such a low titanium content yellow iron oxide precipitate, it is important to ensure that the components used in its manufacture contain sufficiently low concentrations of titanium so that the yellow iron oxide precipitate used as the precursor of the iron oxide component of the inventive catalyst has a low concentration of titanium. Indeed, this is one aspect of the inventive catalyst that is at odds with the prior art teachings, such as those of U.S. Pat. No. 5,190,906, which indicate that titanium may be incorporated into its iron oxide by the introduction of water-soluble titanium salts into an aqueous iron salt solution from which an iron oxide precipitate is formed. This, in fact, is not a desired feature of the invention herein, and it would have the effect of providing a yellow iron oxide precipitate with too high of a titanium content to be useful as a precursor of the red iron oxide component of the inventive catalyst. U.S. Patent Publication 2003/0223942 describes a method of making a high purity yellow iron oxide that may be used to prepare yellow iron oxide, having a low titanium concentration, that is suitable for use in the preparation of the iron oxide component of the inventive catalyst. This publication is hereby incorporated herein by reference.

As earlier noted, it is an important feature of the invention for the iron oxide component of the inventive iron oxide based dehydrogenation catalyst composition to have a low titanium content, such as a concentration of less than about 1500 parts per million by weight (ppmw). But, in general, it is desirable for the titanium concentration of the iron oxide component to be much lower, such as, less than 600 ppmw or even less than 500 ppmw. Preferably, the iron oxide component has a concentration of titanium of less than 250 ppmw, and, most preferably, less than 150 ppmw. There is no particular lower limit for the concentration range of titanium in the iron oxide component except those limitations due to practical considerations. Thus, the lower end of the titanium concentration range in the iron oxide component can be as low as, for example, one part per billion by weight (ppbw). A preferred lower limit for the titanium concentration range in the iron oxide component, however, is one ppmw.

The references herein to the concentration of titanium in, or to the titanium content of, the iron oxide component are referring to the titanium in the elemental form even though the titanium is probably present in the iron oxide component in some other form, such as, as an oxide, e.g. TiO₂. Also, the reference to the titanium concentration measurement in terms of parts by weight is based on the iron oxide component that is derived by the heat treatment of the yellow iron oxide precipitate.

To provide the iron oxide component having the required low titanium content, the precursor yellow iron oxide precipitate should have a low titanium concentration. Thus, in the manufacture of the yellow iron oxide precipitate it is important to use components or raw materials that contain low concentrations of titanium so as to give a yellow iron oxide precipitate for use in the preparation of the iron oxide component of the inventive iron oxide based dehydrogenation catalyst composition having a low titanium concentration of less than about 1500 ppmw. It is desirable, however, for the titanium concentration of the yellow iron oxide precipitate to be less than 1000 ppmw. Preferably, the concentration of titanium in the yellow iron oxide precipitate is less than 500 ppmw, and, most preferably, less than 200 ppmw. The references herein to the concentration of titanium in the yellow iron oxide precipitate refers to the titanium as if it is in the elemental form even though the titanium may actually be present in the yellow iron oxide precipitate in some other form such as an oxide form, e.g. TiO₂.

To prepare the iron oxide component, the yellow iron oxide precipitate is subjected to a heat treatment that converts at least a portion, preferably, a major portion or substantially all, of the yellow iron oxide (FeOOH) to red iron oxide (α-Fe₂O₃). The heat treatment, or calcination, of the yellow iron oxide precipitate can be conducted in the presence of a gaseous atmosphere that includes a gas selected from the group consisting of nitrogen, oxygen, carbon dioxide, air and mixtures of two or more thereof. Preferably, the gaseous atmosphere comprises air.

The temperature range at which the heat treatment is conducted can be any temperature that converts the yellow iron oxide precipitate to an iron oxide that can suitably be used as a component of the inventive catalyst. The heat treatment temperature, thus, can be in the range of from 400° C. to 1000° C., and, preferably, from 500° C. to 950° C. Most preferably, the heat treatment of the yellow iron oxide precipitate is conducted in an atmosphere of air at a temperature in the range of from 600° C. to 900° C.

One method that may be used to prepare the iron oxide component, provided it is able to give an iron oxide having the required low titanium concentration, is described in European patent publication EP 1 388 523, which claims priority to U.S. Pat. No. 212,196. These patent documents are hereby incorporated herein by reference.

The inventive iron oxide based dehydrogenation catalyst, having a low concentration of titanium, is prepared by combining the aforedescribed iron oxide component formed by the heat treatment of a yellow iron oxide precipitate having a low titanium content with at least one additional dehydrogenation catalyst component to form a mixture of the components, shaping the mixture of components into particles, and heat treating or calcining the particles to yield the iron oxide based dehydrogenation catalyst having a low concentration of titanium.

In an alternative preparation method, instead of using the heat treated yellow iron oxide precipitate as an iron oxide component of the mixture, a non-heat treated yellow iron oxide precipitate having a low titanium content is used as a component of the mixture that is formed into particles and then calcined. The calcination of the particles provides for the conversion of the yellow iron oxide to the desired red iron oxide.

In still another alternative preparation method, a combination of both a yellow iron oxide precipitate and a red iron oxide formed by the heat treatment of a yellow iron oxide precipitate, provided that both have the necessary low titanium concentration, are combined with at least one additional dehydrogenation catalyst components to form the mixture of components. This mixture is then formed into particles that are heat treated to yield the iron oxide based dehydrogenation catalyst having a low concentration of titanium.

The particles that are formed in the preparation of the inventive iron oxide based dehydrogenation catalyst can be any type of particle known to those skilled in the art as being a suitable type of agglomerate or shape for use as a catalyst particle, including, for example, extrudates, pellets, tablets, spheres, pills, saddles, trilobes, tetralobes, and the like. One preferred method of making the inventive iron oxide based dehydrogenation catalyst is to mix the components with water, or a plasticizer, or both, and forming an extrudable paste from which extrudates are formed. The extrudates are dried and calcined. The calcination is preferably conducted in an oxidizing atmosphere, such as air, at temperatures upwardly to 1200° C., preferably from 500° C. to 1100° C., and, most preferably, from 700° C. to 1050° C.

The proportions of the components that are mixed together are such as to ultimately provide the inventive iron oxide based dehydrogenation catalyst having an iron oxide content in the range of 10 to upwardly to 98 or even greater weight percent iron oxide, with the weight percent being based on the total weight of the iron oxide based dehydrogenation catalyst composition and calculated as Fe₂O₃. It is preferred, however, for the iron oxide content to be in the range of from 40 to 90 weight percent, and, most preferred, from 60 to 85 weight percent. When red iron oxide and yellow iron oxide are mixed together in the preparation of the inventive iron oxide based dehydrogenation catalyst, any suitable proportion of the two may be mixed together to form the mixture that is agglomerated into particles that are heat treated. Generally, however, the proportion of the yellow iron oxide used in the mixture is in the range of upwardly to about 50 weight percent, preferably, from 0 to 30 weight percent, of the total weight of the iron oxide in the mixture.

The inventive iron oxide based dehydrogenation catalyst may further include an additional dehydrogenation catalyst component, such as, for example, potassium and certain other promoter metals. The potassium and promoter metal or metals are typically present in the form of an oxide. The promoter metal may be selected from the group consisting of Sc, Y, La, Mo, W, Ce, Rb, Ca, Mg, V, Cr, Co, Ni, Mn, Cu, Zn, Cd, Al, Sn, Bi, rare earths and mixtures of any two or more thereof. Among the promoter metals, preferred are those selected from the group consisting of Ca, Mg, Mo, W, Ce, La, Cu, Cr, V and mixtures of any two or more thereof. In addition to the potassium, the most preferred additional dehydrogenation catalyst component is selected from the group consisting of Ca, Mg, W, Mo, Ce, and any two or more thereof.

The potassium (K) component may be present in the iron oxide based dehydrogenation catalyst in the range of from 5 to 40 weight percent with the weight percent being based on the total weight of the iron oxide based dehydrogenation catalyst and calculated as K₂O. Preferably, the potassium is present in the iron oxide based-dehydrogenation catalyst in the range of from 5 to 35 weight percent, and, most preferably, from 10 to 30 weight percent.

Relative to the iron oxide (Fe₂O₃) contained in the finished iron oxide based dehydrogenation catalyst of the invention, the potassium component may be present therein in such an amount as to provide a ratio of the potassium component, calculated as K, to iron oxide that is in the range of from about 200 millimole (mmole) K per mole Fe₂O₃ to about 1600 mmole K per mole Fe₂O₃. Preferably, the potassium to iron oxide ratio is in the range of from 200 mmole K/mole Fe₂O₃ to 1400 mmole K/mole Fe₂O₃, and, most preferably, from 400 mmole K/mole Fe₂O₃ to 1200 mmole K/mole Fe₂O₃.

The alkaline earth metal component of the iron oxide based dehydrogenation catalyst can include either a magnesium (Mg) component or a calcium (Ca) component, or both components, and each such component alone or in combination may be present in the iron oxide based dehydrogenation catalyst in the range of from 0.1 to 15 weight percent with the weight percent being based on the total weight of the iron oxide based dehydrogenation catalyst and with the alkaline earth metal calculated as an oxide. Preferably, the alkaline earth metal is present in the iron oxide based dehydrogenation catalyst in the range of from 0.2 to 10 weight percent, and, most preferably, from 0.3 to 5 weight percent.

Relative to the iron oxide (Fe₂O₃) contained in the finished iron oxide based dehydrogenation catalyst of the invention, the alkaline earth metal component may be present therein in such an amount as to provide a ratio of the alkaline earth metal component, calculated as the element, to iron oxide that is in the range of from about 5 millimole (mmole) alkaline earth metal per mole Fe₂O₃ to 750 mmole alkaline earth metal per mole Fe₂O₃. Preferably, the alkaline earth metal to iron oxide ratio is in the range of from 10 mmole alkaline earth metal/mole Fe₂O₃ to 500 mmole alkaline earth metal/mole Fe₂O₃, and, most preferably, from 15 mmole alkaline earth metal/mole Fe₂O₃ to 250 mmole alkaline earth metal/mole Fe₂O₃.

The cerium (Ce) component may be present in the iron oxide based dehydrogenation catalyst in the range of from 1 to 25 weight percent with the weight percent being based on the total weight of the iron oxide based dehydrogenation catalyst and calculated as CeO₂. Preferably, the cerium is present in the iron oxide based dehydrogenation catalyst in the range of from 2 to 20 weight percent, and, most preferably, from 3 to 15 weight percent.

Relative to the iron oxide (Fe₂O₃) contained in the finished iron oxide based dehydrogenation catalyst of the invention, the cerium component may be present therein in such an amount as to provide a ratio of the cerium component, calculated as Ce, to iron oxide that is in the range of from about 14 millimole (mmole) Ce per mole Fe₂O₃ to 350 mmole Ce per mole Fe₂O₃. Preferably, the cerium to iron oxide ratio is in the range of from 28 mmole Ce/mole Fe₂O₃ to 280 mmole Ce/mole Fe₂O₃, and, most preferably, from 42 mmole Ce/mole Fe₂O₃ to 200 mmole Ce/mole Fe₂O₃.

The molybdenum (Mo) component or tungsten (W) component, or both components, and each such component alone or in combination, may be present in the iron oxide based dehydrogenation catalyst in the range of from 0.1 to 15 weight percent with the weight percent being based on the total weight of the iron oxide based dehydrogenation catalyst and calculated as either MoO₃ or WO₃, dependent upon the component considered. Preferably, the molybdenum or tungsten, or both, is present in the iron oxide based dehydrogenation catalyst in the range of from 0.2 to 10 weight percent, and, most preferably, from 0.3 to 5 weight percent.

Relative to the iron oxide (Fe₂O₃) contained in the finished iron oxide based dehydrogenation catalyst of the invention, the molybdenum or tungsten component may be present therein in such an amount as to provide a ratio of the molybdenum or tungsten component, respectively calculated as Mo or W, to iron oxide that is in the range of from about 2 millimole (mmole) Mo or W per mole Fe₂O₃ to 300 mmole Mo or W per mole Fe₂O₃. Preferably, the molybdenum to iron oxide ratio is in the range of from 4 mmole Mo or W/mole Fe₂O₃ to 200 mmole Mo or W/mole Fe₂O₃, and, most preferably, from 6 mmole Mo or W/mole Fe₂O₃ to 100 mmole Mo or W/mole Fe₂O₃.

A preferred iron oxide based dehydrogenation catalyst composition of the invention has a low titanium concentration or an absence of or a substantial absence of titanium and comprises from 40 to 90 weight percent of the iron oxide component, as described herein, calculated as Fe₂O₃, and from 5 to 40 weight percent of a potassium component, calculated as K₂O. The iron oxide based dehydrogenation catalyst composition can further comprise from 1 to 25 weight percent of a cerium component, calculated as CeO₂; and, it further can comprise from 0.1 to 15 weight percent of a molybdenum component, calculated as MoO₃; and, it further can comprise from 0.1 to 15 weight percent an alkaline earth metal component, calculated as an oxide.

Another preferred iron oxide based dehydrogenation catalyst composition has a low titanium concentration or an absence of or a substantial absence of titanium and further comprises from 40 to 90 weight percent of the iron oxide component, as described herein and calculated as Fe₂O₃, and a potassium component present in the iron oxide based dehydrogenation catalyst composition in such an amount as to provide a ratio of the potassium component, calculated as K, to iron oxide that is in the range of from about 200 millimole (mmole) K per mole Fe₂O₃ to about 1600 mmole K per mole Fe₂O₃. The iron oxide based dehydrogenation catalyst composition can further comprise a cerium component present in the iron oxide based dehydrogenation catalyst composition in such an amount as to provide a ratio of the cerium component, calculated as Ce, to iron oxide that is in the range of from about 14 millimole (mmole) Ce per mole Fe₂O₃to 350 mmole Ce per mole Fe₂O₃; and it further can comprise a molybdenum component present in such an amount as to provide a ratio of the molybdenum component, calculated as Mo, to iron oxide that is in the range of from about 2 millimole (mmole) Mo per mole Fe₂O₃ to 300 mmole Mo per mole Fe₂O₃; and it further can comprise an alkaline earth metal component present in such an amount as to provide a ratio of the alkaline earth metal component, calculated as the element, to iron oxide that is in the range of from about 5 millimole (mmole) alkaline earth metal per mole Fe₂O₃to 750 mmole alkaline earth metal per mole Fe₂O₃.

Another preferred iron oxide based dehydrogenation catalyst of the invention has a low titanium content and consists essentially of the iron oxide component, as described herein, a potassium component, a cerium component, a molybdenum or tungsten component, and an alkaline earth component.

As already noted, an important feature of the invention is for the inventive iron oxide based dehydrogenation catalyst to have a low titanium concentration, and, to maximize stability, the titanium concentration should be less than 900 ppmw. The titanium content of the yellow iron oxide precipitate has the most, if not the only, influence on the concentration of titanium that is in the inventive iron oxide based dehydrogenation catalyst. Thus, the titanium concentration of the inventive iron oxide based dehydrogenation catalyst is, in most instances, controlled by the titanium concentration in the yellow iron oxide used in the preparation of the inventive catalyst, which should have the titanium concentration as described above. In any event, it is desirable for the titanium concentration of the inventive iron oxide based dehydrogenation catalyst to be less than 300 ppmw, and, preferably, less than 150 ppmw. Most preferably, the titanium concentration is less than 125 ppmw. It is especially preferred for the titanium concentration of the inventive iron oxide based dehydrogenation catalyst to be less than 50 ppmw and, most especially preferred, less than 30 ppmw.

The inventive catalysts described herein may suitably be used in the dehydrogenation of dehydrogenatable hydrocarbons, and, further, they may be used to improve the operation of existing dehydrogenation reactor systems that include a dehydrogenation reactor vessel in which is contained a volume of a dehydrogenation catalyst having either a high titanium content that causes it to have unfavorable stability properties relative to the inventive catalysts or an activity reducing concentration of titanium, or both. It is understood that the high titanium content that causes unacceptable catalytic properties of a dehydrogenation catalyst, or that is activity reducing, includes concentration ranges greater than those described elsewhere herein as being a low concentration of titanium. The operation of the existing dehydrogenation reactor system is improved by replacing such low stability dehydrogenation catalyst with the inventive catalyst. In the dehydrogenation method, the inventive catalyst is contacted with a dehydrogenation feed under dehydrogenation reaction conditions to thereby provide a dehydrogenation reaction product. More specifically, the dehydrogenation feed is introduced into the dehydrogenation reactor wherein it is contacted with the dehydrogenation catalyst bed.

It is recognized that the dehydrogenation reactor or dehydrogenation reactor system can include more than one dehydrogenation reactor or reaction zone. If more than a single dehydrogenation reactor is used, they may be operated in series or in parallel, or they may be operated independently from each other or under the same or different process conditions.

The dehydrogenation feed can be any suitable feed and, more particularly, it can include any hydrocarbon that is dehydrogenatable. Examples of dehydrogenatable hydrocarbons include alkyl aromatics, such as alkyl substituted benzene and alkyl substituted naphthalene, isoamylenes, which can be dehydrogenated to isoprenes, and butenes, which can be dehydrogenated to butadiene. The preferred dehydrogenation feed comprises an alkylaromatic compound preferably one selected from the group of compounds consisting of ethylbenzene, propylbenzene, butylbenzene, hexylbenzene, methylpropylbenzene, methylethylbenzene, and diethylbenzene. The most preferred dehydrogenation feed is an ethylbenzene feedstock comprising, other than diluents such as steam, predominantly ethylbenzene. Ethylbenzene is dehydrogenated to styrene. The dehydrogenation feed can also include other components including diluents. It is common to use steam as a feed diluent when ethylbenzene is a feed component to be dehydrogenated to form styrene.

The dehydrogenation conditions can include a dehydrogenation reactor inlet temperature in the range of from about 500° C. to about 1000° C., preferably, from 525° C. to 750° C., and, most preferably, from 550° C. to 700° C. It is recognized that in the dehydrogenation of ethylbenzene to styrene the reaction is endothermic. When such a dehydrogenation reaction is carried out, it can be done so either isothermally or adiabatically. In the case where the dehydrogenation reaction is carried out adiabatically, the temperature across the dehydrogenation catalyst bed, between the dehydrogenation reactor inlet and the dehydrogenation reactor outlet, can decrease by as much as 150° C., but, more typically, the temperature can decrease from 10° C. to 120° C.

The reaction pressure is relatively low and can range from vacuum pressure upwardly to about 200 kPa (29 psi). Typically, the reaction pressure is in the range of from 20 kPa absolute (2.9 psia) to 200 kPa (29 psi). Due to the reaction kinetics of the dehydrogenation reaction of ethylbenzene to styrene it is generally preferable for the reaction pressure to be as low as is commercially feasible.

The liquid hourly space velocity (LHSV) can be in the range of from about 0.01 hr⁻¹ to about 10 hr⁻¹, and preferably, from 0.1 hr⁻¹ to 2 hr⁻¹. As used herein, the term “liquid hourly space velocity” is defined as the liquid volumetric flow rate of the dehydrogenation feed, for example, ethylbenzene, measured at normal conditions (i.e., 0° C. and 1 bar absolute), divided by the volume of the catalyst bed, or the total volume of catalyst beds if there are two or more catalyst beds. When styrene is being manufactured by the dehydrogenation of ethylbenzene, it generally desirable to use steam as a diluent usually in a molar ratio of steam to ethylbenzene in the range of 0.1 to 20. Typically, the molar ratio of steam to ethylbenzene is in the range of from 2 to 15, and, more typically, from 4 to 12. Steam may also be used as a diluent with other dehydrogenatable hydrocarbons.

The following Examples are presented to illustrate the invention, but they should not be construed as limiting the scope of the invention.

### EXAMPLE I

This Example I describes the preparation of several iron oxide based dehydrogenation catalysts having varying concentrations of titanium. These catalysts were tested for their performance and stability as presented in Example II.

Three Catalysts A, B, and C were prepared first by forming a paste made by mixing the following ingredients: red iron oxide made by the heat treatment of a precipitated yellow iron oxide, potassium carbonate, cerium carbonate, molybdenum trioxide, calcium carbonate, and water (about 10 wt. %, relative to the total weight of the dry mixture). The paste was then extruded to form 3-mm diameter cylinders that were cut into 6-mm lengths to form pellets. These pellets were then dried in air at 170° C. for 15 minutes and subsequently calcined in air at 825° C. for 1 hour. The red iron oxide used to make Catalyst A was formed by the heat treatment of a precipitated yellow iron oxide, made by the Penniman process, but having a low concentration of titanium of about 84 ppm, and, thus, the resulting red iron oxide also contained a low titanium concentration. The red iron oxide used to make Catalysts B and C was formed by the heat treatment of a precipitated yellow iron oxide, made by the Penniman process, having a high concentration of titanium of about 310 ppm. The resulting Catalysts A, B, and C nominally contained per mole of iron oxide, calculated as Fe₂O₃, about 0.516 mole potassium, 0.066 mole cerium, 0.022 mole molybdenum, and 0.027 mole calcium. Catalyst A had a low concentration of titanium as compared to the concentration of titanium in Catalysts B and C. Catalyst A contained a titanium concentration of about 96 ppm. Catalysts B and C respectively contained a titanium concentration of about 255 ppm and 287 ppm.

### EXAMPLE II

The Example II describes the procedure for testing the performance of the catalysts described in Example I and presents the results of such testing.

Samples of Catalysts A, B, and C were tested for their performance in the dehydrogenation manufacturing of styrene from ethylbenzene under isothermal testing conditions in a reactor designed for continuous operation. A sample of each of the three catalyst samples was individually tested under the following testing conditions: absolute pressure 76 kPa, steam to ethylbenzene molar ratio 10, LHSV 0.65 l/I.h. In each test run, the reactor temperature was set at 600° C. for the first three days, and, then, the reactor temperature was adjusted daily such that in each test run a 70% mole conversion of ethylbenzene was achieved.

Presented in Table 1 is summary data from the testing of the three catalyst samples.

TABLE 1
Summary Performance Data from the
testing of Catalysts A, B, and C.

| Catalyst A | | | Catalyst B | | | Catalyst C | | |
|---|---|---|---|---|---|---|---|---|
| | T(70) | S(70) | | T(70) | S(70) | | T(70) | S(70) |
| Day | ° C. | % | Day | ° C. | % | Day | ° C. | % |
| 1.3 | 597.0 | 93.4 | 0.9 | 586.5 | 93.2 | 0.9 | 586.7 | 93.3 |
| 2.3 | 590.1 | 93.5 | 1.9 | 586.7 | 93.1 | 1.9 | 587.1 | 93.0 |
| 3.3 | 590.1 | 93.7 | 2.9 | 587.3 | 93.4 | 2.9 | 587.6 | 93.3 |
| 4.3 | 590.9 | 94.9 | 3.9 | 589.6 | 94.7 | 3.9 | 588.8 | 94.3 |
| 5.3 | 591.1 | 95.0 | 4.9 | 590.1 | 94.9 | 4.9 | 590.3 | 94.6 |
| 6.3 | 590.3 | 95.1 | 5.9 | 591.9 | 95.0 | 5.9 | 592.2 | 94.8 |
| 7.3 | 589.1 | 95.0 | 6.9 | 592.8 | 95.1 | 6.9 | 593.7 | 94.9 |
| 8.3 | 589.6 | 95.0 | 8.2 | 595.1 | 95.1 | 8.1 | 595.2 | 95.0 |
| 9.3 | 590.2 | 95.1 | 9.1 | 596.2 | 95.2 | 9.1 | 597.2 | 95.0 |
| 10.3 | 590.8 | 95.2 | 9.9 | 597.1 | 95.1 | 9.9 | 598.2 | 95.0 |
| 11.5 | 590.6 | 95.3 | 10.9 | 596.8 | 95.1 | 10.9 | 599.0 | 95.0 |
| 12.3 | 590.9 | 95.3 | 11.9 | 597.5 | 95.1 | 11.9 | 599.1 | 94.9 |
| 13.3 | 591.3 | 95.2 | 12.9 | 597.7 | 95.1 | 12.9 | 600.4 | 94.9 |
| | | | 13.9 | 598.9 | 95.1 | 13.9 | 601.5 | 94.9 |
| | | | 14.9 | 598.8 | 95.1 | 14.9 | 601.5 | 95.0 |
| | | | 15.9 | 599.5 | 95.0 | 15.9 | 602.5 | 95.1 |
| | | | 16.9 | 600.4 | 95.1 | 16.9 | 603.5 | 94.9 |
| | | | 17.9 | 600.7 | 95.0 | 17.9 | 603.5 | 94.9 |
| | | | 18.9 | 601.8 | 95.0 | 18.9 | 604.8 | 94.8 |
| | | | 19.9 | 601.9 | 95.0 | | | |

As may be seen from the data presented in Table 1, and FIG. 1 Catalyst A showed very little decline in activity (T70) from day 3-13, while Catalysts B and C showed activity decline of over 10° C. in the same time period. Moreover, the activity of Catalysts B and C continued to decline in a linear fashion for the remainder of the test period and at a rate that was significantly greater than the decline in activity for Catalyst A. It also should be noted that Catalyst A exhibited comparable or somewhat better selectivity at a 70 percent conversion, i.e. S(70), than Catalysts B and C.

FIG. 1 is provided to further illustrate and to assist in an appreciation of the advantages of the invention. FIG. 1 presents plots of the activity of each of the three catalysts as a function of time in use. As may be observed from the plots, Catalyst A, which contains a low titanium concentration, exhibited a significantly lower rate of deactivation with use than Catalysts B and C, both of which have a high concentration of titanium. While Catalysts B and C may have exhibited, as demonstrated by a lower T(70) temperature, somewhat of a higher initial activity than that of Catalyst A, after a short period of use, Catalyst A with its lower titanium concentration exhibited a significantly higher activity than that of Catalysts B and C that contained a higher titanium concentration. This is attributable to the apparently greater stability of the low titanium containing catalyst.

Reasonable variations, modifications and adaptations of the invention may be made within the scope of the described disclosure and appended claims without departing from the spirit and scope of the invention.

That which is claimed is:

1. An iron oxide based dehydrogenation catalyst composition, comprising:
an iron oxide component having a low titanium content, wherein said iron oxide component is made by heat-treating of a yellow iron oxide precipitate made by precipitating from a solution of an iron salt said yellow iron oxide precipitate, and wherein said iron oxide based dehydrogenation catalyst composition has a first concentration of titanium.

2. A composition as recited in claim 1, wherein said low titanium content of said iron oxide component is such as to provide said first concentration of titanium that is less than about 300 ppm.

3. A composition as recited in claim 2, wherein said yellow iron oxide precipitate has a second concentration of titanium that is less than about 300 ppm.

4. A composition as recited in claim 3, wherein said yellow iron oxide precipitate further is defined as being formed by adding a colloidal hydrate of iron to an aqueous solution of an iron salt, wherein contained within said aqueous solution is metallic iron; introducing an oxidizing agent into said aqueous solution; and precipitating and recovering said yellow iron oxide precipitate.

5. A composition as recited in claim 4, wherein said iron oxide based dehydrogenation catalyst composition further includes an additional dehydrogenation catalyst component selected from the group of compounds consisting of potassium compounds, cerium compounds, molybdenum compounds, tungsten compounds and calcium compounds.

6. A composition as recited in claim 5, wherein said iron oxide based dehydrogenation catalyst composition further comprises an amount of iron oxide in the range of from 10 weight percent to 98 weight percent, wherein the weight percent is based on the total weight of said iron oxide based dehydrogenation catalyst composition and calculated as Fe₂O₃.

7. A composition as recited in claim 6, wherein said heat treatment is conducted in an atmosphere comprising air at a temperature in the range of from 400° C. to 1000° C.

8. A composition as recited in claim 7, wherein said first concentration of titanium is less than 250 ppmw.

9. A method of making an iron oxide based dehydrogenation catalyst having a first concentration of titanium, wherein said method comprises:
forming a red iron oxide component, having a low titanium content, by heat-treating a yellow iron oxide precipitate made by the precipitation thereof from a solution of an iron salt, wherein said yellow iron oxide has a second concentration of titanium;
mixing said red iron oxide component with an additional dehydrogenation catalyst component and water to form a mixture;
forming a particle from said mixture; and
heat-treating said particle to thereby provide said iron oxide based dehydrogenation catalyst.

10. A method as recited in claim 9, wherein said low titanium content of said iron oxide component is such as to provide said first concentration of titanium that is less than about 300 ppm.

11. A method as recited in claim 10, wherein said yellow iron oxide precipitate has a second concentration of titanium that is less than about 300 ppm.

12. A method as recited in claim 11, wherein said yellow iron oxide precipitate further is defined as being formed by adding a colloidal hydrate of iron to an aqueous solution of an iron salt, wherein contained within said aqueous solution is metallic iron; introducing an oxidizing agent into said aqueous solution; and precipitating and recovering said yellow iron oxide precipitate.

13. A method as recited in claim 12, wherein said additional dehydrogenation catalyst component is selected from the group of compounds consisting of potassium compounds, cerium compounds, molybdenum compounds, tungsten compounds, alkaline earth metal compounds, and calcium compounds.

14. A method as recited in claim 13, wherein said iron oxide based dehydrogenation catalyst composition further comprises an amount of iron oxide in the range of from 10 weight percent to 98 weight percent, wherein the weight percent is based on the total weight of said iron oxide based dehydrogenation catalyst composition and calculated as Fe₂O₃.

15. A method as recited in claim 14, wherein said heat treatment of said yellow iron oxide precipitate is conducted in an atmosphere comprising air at a temperature in the range of from 400° C. to 1000° C.

16. A method as recited in claim 15, wherein said first concentration of titanium is less than 250 ppmw.

17. A method as recited in claim 16, wherein said second concentration of titanium is less than 250 ppmw.

18. A dehydrogenation process, comprising:
contacting under dehydrogenation conditions a dehydrogenatable hydrocarbon with an iron oxide based dehydrogenation catalyst composition that comprises an iron oxide component having a low titanium content, wherein said iron oxide component is made by heat-treating of a yellow iron oxide precipitate made by precipitating from a solution of an iron salt said yellow iron oxide precipitate and an additional dehydrogenation catalyst component, wherein said iron oxide based dehydrogenation catalyst composition has a first concentration of titanium; and
yielding a dehydrogenation product.

19. A dehydrogenation process, comprising contacting under dehydrogenation conditions a dehydrogenatable hydrocarbon with the catalyst of claim 1 and yielding a dehydrogenation product.

20. A catalyst composition made by the method of claim 1.

21. A method of improving the operation of a dehydrogenation reactor system, which comprises a dehydrogenation reactor containing a first volume of a dehydrogenation catalyst having an activity reducing concentration of titanium, wherein said method comprises:
removing from said dehydrogenation reactor said dehydrogenation catalyst and replacing therewith an iron oxide based dehydrogenation catalyst composition that comprises an iron oxide component having a low titanium content, wherein said iron oxide component is made by heat-treating of a yellow iron oxide precipitate made by precipitating from a solution of an iron salt said yellow iron oxide precipitate and an additional dehydrogenation catalyst component, wherein said iron oxide based dehydrogenation catalyst composition has a first concentration of titanium, to thereby provide a second dehydrogenation reactor system; and
operating said second dehydrogenation reactor system under dehydrogenation reaction conditions.
